(19) United States
(12) Patent Application Publication
(10) Pub. No.: US 2004/0043026 A1
(43) Pub. Date: Mar. 4, 2004

(54) Treatment and prevention of abnormal scar formation in keloids and other cutaneous or internal wounds or lesions

(76) Inventors: Tai-Lan Tuan, Fullerton, CA (US); Paul D. Benya, Los Angeles, CA (US); David Warburton, La Canada, CA (US)

Correspondence Address: PERKINS COIE LLP, POST OFFICE BOX 1208, SEATTLE, WA, 98111-1208, US

(21) Appl. No.: 10/439,267

(22) Filed: May 13, 2003

Related U.S. Application Data
(60) Provisional application No. 60/380,696, filed on May 13, 2002.

(51) Int. Cl. A61K 39/395; A61K 38/05; A61K 31/58; A61K 31/56; A61K 31/495; A61K 31/355; A61K 31/401

(52) U.S. Cl. 424/146.1; 514/174; 514/179; 514/423; 514/18; 514/458; 514/474; 514/255.02; 514/560; 514/312

(57) ABSTRACT

The present invention relates to findings that reducing the activity of Plasminogen Activator Inhibitor-1 (PAI-1) suppresses an excessive deposition of collagen which is known as a cause for the formation of abnormal scars. These abnormal scars include but are not limited to keloids, adhesions, hypertrophic scars, skin disfiguring conditions, fibrosis, fibrocystic conditions, contractures, and scleroderma, all of which are associated with or caused by an excessive deposit of collagen in a wound healing process. Accordingly, aspects of the present invention are directed to the reduction of PAI-1 activity to decrease an excessive accumulation of collagen, prevent the formation of an abnormal scar, and/or treat abnormal scars that result from an excessive accumulation of collagen. The PAI-1 activity can be reduced by PAI-1 inhibitors which include but are not limited to PAI-1 neutralizing antibodies, diketopiperazine based compounds, tetramic acid based compounds, hydroxyquinolinone based compounds, Enalapril, Eprosartan, Troglitazone, Vitamin C, Vitamin E, Mifepristone (RU486), and Spironolactone to name a few. Another aspect of the present invention is directed to methods of measuring PAI-1 activity in a wound healing process and determining the propensity of the formation of an abnormal scar.

### RELATED APPLICATION

This application claims the benefit of U.S. Provisional Application No.60/380,696, filed May 13, 2002, which is hereby incorporated by reference in its entirety including drawings as fully set forth herein.

### REFERENCE TO GOVERNMENT GRANT

The present invention was made with government support under grant GM 55081 by the National Institute of General Medical Sciences. The U.S. government may have certain rights in this invention.

### FIELD OF THE INVENTION

The present invention relates to the treatment or prevention of abnormal scar formation. Specifically, the present invention relates to the reduction of the activity of plasminogen activator inhibitor-1 to decrease an excessive deposit of collagen in a wound healing process that causes abnormal scars including keloids, hypertrophic scars, adhesions, and other cutaneous or internal wounds or lesions.

### BACKGROUND OF THE INVENTION

Wound healing is a continuous process commonly divided into four separate phases: 1) coagulation, 2) inflammation, 3) migration and proliferation, and 4) remodeling.

Soon after a wound occurs in a subject, the wound healing process starts with a coagulation of fibrin and fibronectin to form a matrix or a clot and a gathering of platelets at the wound site. As the platelets coagulate, inflammatory cells, such as neutrophils, lymphocytes, and macrophages, are also attracted to the wound site and release factors for wound healing. For example, macrophages secrete cytokines and growth factors such as fibroblast growth factors (FGF), platelet-derived growth factors (PDGF), tumor necrosis growth factors (TNF-α), vascular endothelial growth factors (VEGF), interleukin-1 (IL-1), interferon-gamma (INF-γ); and an epidermal growth factor-like substance. Activated platelets also release epidermal growth factor (EGF), PDGF, transforming growth factors α, β1, and β2 (TGF-α, TGF-α, and TGF-β, respectively); platelet derived epidermal growth factor (PDEGF), platelet-activating factor (PAF), insulin-like growth factor-1 (INF-1), fibronectin, and serotonin. Together these biological factors are involved in the infiltration, proliferation, and migration of keratinocytes, fibroblasts, and endothelial cells. Towards the end of the inflammation phase, proteins, fats, and cross-linked new collagen aggregate together and form a transient scaffold.

During the migration and proliferation phase, cells that have migrated into the wound site undergo rapid mitosis and differentiation. These cells include keratinocytes and fibroblasts. On one hand, keratinocytes undergo an epithelization process in which the cells stratify and differentiate to form an epidermal covering. Keratinocytes also release keratinocyte growth factor (KGF) and VEGF to stimulate angiogenesis, TGF-α as a chemoattractant, PDGF to promote extracellular matrix (ECM) formation, and proteases to dissolve nonviable tissue and fibrin barriers. Migrated fibroblasts, on the other hand, synthesize and deposit collagen and proteoglycans, release growth factors such as KGF, connective tissue growth factors (CTGF), plasminogen activator inhibitor-1 (PAI-1) and TGF-β. Like the keratinocytes, fibroblasts also release proteases that expedite the subsequent remodeling process. All these cellular activities such as migration, proliferation, differentiation, degradation of the transient scaffold, and synthesis of a new matrix in the migration and proliferation phase are often described as a fibroplasia process.

The final stage of wound healing is involved in a remodeling process which changes the deposition pattern of matrix components. As described, the initial matrix is a clot of fibrin and fibronectin resulting from homeostasis. With the proliferation and migration of fibroblasts, collagen is synthesized and deposited replacing and rearranging the initial matrix with aid from proteases. Collagen fibers gradually increase in thickness and align along the stress line of the wound. At the end of an normal scar formation, the final scar shows collagen fibers mostly parallel to the epidermis. (For reviews, See, Hunt et al., Physiology of Wound Healing, Adv. Skin Wound Care 13: 6-11 (2000); Ferguson et al., Scar Formation: The Special Nature of Fetal and Adult Wound Repair, Plas. Reconstr. Surg. 97: 854-60 (1996); Gailit & Clark, Wound Repair in the Context of Extracellular Matrix, Curr. Opin. Cell Biol. 6: 717-25 (1994)).

Thus, the wound healing process is a delicately balanced equilibrium between growth and degradation. Any aberrations in the process may tip the balance toward a pathological abnormality in wound healing or an excessive deposit of scarring tissues. For example, an excessive deposition of scar tissues in skin during a wound healing process may result in, for example, keloids or hypertrophic scars. Keloids are a disorder in wound healing wherein excessive scar tissue proliferates beyond the boundary of the original wound. In contrast, hypertrophic scars occur when a trauma or injury to the deep dermis; however, the excessive deposition of scar tissue is confined to the margin of the original wound. In both cases, over accumulation or expression of collagen is believed to be the cause. Tuan & Nichter, The Molecular Basis of Keloid and Hypertrophic Scar Formation, Mol. Med. Today 4: 19-24 (1998).

The presence of abnormally formed scar on skin is frequently cosmetically unacceptable to the affected individual. As a matter of fact, therapeutic strategies to avert or treat abnormalities in wound healing or abnormal scars are one of the driving forces in the cosmetic industry. Additionally, abnormal scars may be painful or pruritic and may restrict certain ranges of motion. In severe cases, it may lead to dysfunction of tissues or organs when wounds occur. Thus, abnormalities in wound healing and abnormal scars warrant clinical investigations and medical treatments.

The cellular and molecular etiology of abnormal scar formation is a subject under intensive investigation. Researches have shown that growth factors are involved in the pathogenesis of abnormal scar formation. In particular, members of TGF-β family play important biological roles. It is reported that TGF-β1 and TGF-β2 are identified at higher levels in keloid fibroblast cultures compared with normal dermal fibroblast cultures and therefore are associated with abnormal scar formation and fibrosis. Lee et al., Expression of Transforming Growth Factor β1, 2, and 3 Proteins in Keloids, Ann. Plast. Surg. 43: 179-184 (1999).

Conventional prevention or treatments for abnormally formed scars include direct corticosteriod injection into a wound site to inhibit fibroblast growth, silicone gel sheeting to treat pruritus associated with keloids, cyrotherapy to cause thermal injury or death of keloids, surgical excision to remove the overgrown scar tissue, and interferon therapy with the use of IFN-α, IFN-β, and IFN-γ to inhibit collagen synthesis by reducing the synthesis of cellular messenger ribonucleic acids in dermal fibroblasts. However, these treatments have shown severe side effects or the recurrence of abnormal scars since the underlying causes for pathological scar formation are unrecognized. So far, there are no universally accepted treatments that would result in the remission or prevention of abnormal scars. Alster & West, Treatment of Scars: A Review, Ann. Plast. Surg. 39: 418-432 (1995).

Therefore, there continues to be a need for novel methods for treating or preventing abnormalities in wound healing or abnormal scars.

### SUMMARY OF INVENTION

One aspect of the present invention is directed to methods for reducing an excessive accumulation or deposit of collagen in a wound healing process that may lead to the formation of an abnormal scar comprising the step of reducing the activity of plasminogen activator inhibitor-1 (PAI-1).

Another aspect of the present invention is directed to methods for preventing the formation of an abnormal scar comprising the step of reducing the activity of PAI-1.

Yet another aspect of the present invention is directed to methods for treating an abnormal scar through the reduction of the activity of PAI-1.

Yet another aspect of the present invention is directed to methods for determining the propensity of forming an abnormal scar in a wound healing process by measuring the level of PAI-1 activity.

In one embodiment of the invention, the activity of PAI-1 is reduced by a PAI-1 inhibitor. The examples of the PAI-1 inhibitors include but are not limited to Fosinopril; Imidapril; Captopril; Enalapril; L158,809; Eprosartan; Troglitazone; Vitamin C; Vitamin E; Perindorpril; Mifepristone (RU486); Spironolactone; reactive center loop peptides; PAI-1 neutralizing antibodies; diketopiperazine based compounds; tetramic acid based compounds, hydroxyquinolinone based compounds; and 11-keto-9(E), 12(E)-octadecadienoic acid.

In another embodiment of the present invention, a PAI-inhibitor is administered to a subject through an administration route, including but not limited to, oral, enteral, buccal, nasal, topical, rectal, vaginal, aerosol, transmucosal, epidermal, transdermal, ophthalmic, pulmonary, and/or parenteral administration.

In another embodiment of the present invention, the PAI activity is measured by, for example, Chromogenic Assay, Enzyme-Linked Immunosorbent Assay, Fibrin Overlay Assay, and Reverse Fibrin Overlay Assay.

In another embodiment of the invention, an abnormal scar is an abnormality in a wound healing process that results from an excessive accumulation of collagen. Examples of abnormal scars include but are not limited to a keloid, a surgical adhesion, a hypertrophic scar, a skin disfiguring skin such as acne and wrinkling, cellulite formation, neoplastic fibrosis, a fibrosis, a fibrocystic condition, a contracture, a scleroderma, a Duypuytren's disease, a Peyronie's disease, and a joint stiffness.

### BRIEF DESCRIPTION OF THE DRAWING

The accompanying figures of the drawing are incorporated into and form a part of the specification to provide illustrative examples of the present invention and explain the principles of the invention. The figures of the drawing are only for purposes of illustrating preferred and alternate embodiments of how the invention can be made and used. It is to be understood, of course, that the drawing is intended to represent and illustrate the concepts of the invention. The figures of the drawing are not to be construed as limiting the invention to only the illustrated and described examples. Various advantages and features of the present invention will be apparent from consideration of the written specification and the accompanying figures of the drawing wherein:

FIG. 1 shows the immunohistochemistry study of uPA and PAI-1 expressions in normal skin, normal scar, and keloid. Keloid and normal skin samples were from African-American patients where melanocytes appeared dark brown in immunohistochemistry. Ctrl: control without primary antibodies. “*”: Epidermis. “J”, “k”, and “1” are from deep dermal regions of keloid scar. Solid arrow heads in panels “b” and “c” indicate blood vessels. Open arrow heads in panels “h”, “k”, “i”, and “1” indicate fibroblasts. Photo images were taken at 100× magnification.

FIG. 2 shows Northern blot analysis of messenger RNA of PAI-1 from fibroblasts of normal skin, normal scar, or keloid origins. Normal skin (N65 and N77), normal scar (NS70 and NS75), and keloid (K76 and K80) fibroblasts were cultured at a density of 8×10³ cells/cm² and extracted for Northern blot analysis. Twenty μg of total RNA was loaded on each lane. Samples were standardized to the level of β-actin.

FIG. 3 shows a time course study of collagen accumulation comparing normal and keloid fibroblasts cultured in fibrin gels over a 16-day period. Collagen synthesized by either normal or keloid fibroblasts was purified according to the procedure described herein. The amount of purified collagen at each time point is expressed in cpm/cell.

FIG. 4 shows the expressions of uPA and PAI-1 over a 14-day period comparing normal and keloid fibroblasts. Upper panels: fibrin overlay assay demonstrating uPA activities. Lower Panels: reverse fibrin overlay assay demonstrating PAI-1 activities. The two-chain uPA is present with a molecular weight of ˜50 kD. The single-chain uPA is present with a molecular weight of ˜30 kD. The high molecular weight proteins (˜110 kD) are uPA/PAI-1 complexes. Human PAI-1 shows a molecular weight of around 50 kD.

FIG. 5 shows the expressions of uPA and PAI-1 from donor- and anatomical site-matched normal (N86) and keloid (K86) fibroblasts over a 13-day culture period. Upper panels: fibrin overlay assay demonstrating uPA activities. Lower Panels: reverse fibrin overlay assay demonstrating PAI-1 activities. The two-chain uPA is present with a molecular weight of ˜50 kD. The single-chain uPA is present around a molecular weight of ˜30 kD. The high molecular weight proteins (˜110 kD) are uPA/PAI-1 complexes. Human PAI-1 shows a molecular weight of around 50 kD.

FIG. 6 shows the expressions of uPA and PAI-1 of normal or keloid fibroblasts cultured in fibrin, fibrin-collagen, or collagen gels. Upper panels: fibrin overlay assay demonstrating uPA activities. Lower Panels: reverse fibrin overlay assay demonstrating PAI-1 activities. The two-chain uPA is present with a molecular weight of ˜50 kD. The single chain uPA is present around the molecular weight of 30 kD. Human PAI-1 shows a molecular weight of around 50 kD.

FIG. 7 shows the collagen accumulation of normal or keloid fibroblasts cultured in fibrin or collagen gels. Collagen synthesized by fibroblasts was purified as described herein and expressed as cpm/cell.

FIG. 8 shows the effect of anti-PAI-1 neutralizing antibodies on collagen accumulation of keloid fibroblasts cultured in fibrin gels. Collagen synthesized by fibroblasts was purified according to the procedure as described herein and expressed as cpm/cell. Insert: Reverse fibrin overlay demonstrating PAI-1 activity.

FIG. 9 shows a schematic diagram summarizing the major findings of keloid fibrosis and connecting them to key events/components of tissue injury repair. The plasminogen activator/plasmin and PAI-1 system is central to matrix remodeling. It regulates fibrin degradation, influences TGF-beta and matrix metalloproteinase (MMP) activities, and modulates cell adhesion/migration to extracellular matrix (ECM). Keloid fibroblasts exhibited not only an elevated level of collagen accumulation, which could be further increased upon exposure to TGF-β, but also a defect in fibrin degradation which attributed to their increased PAI-1 and decreased uPA activities. The connection between increased PAI-1 activity and excessive collagen accumulation of keloid fibroblasts was established when PAI-1 neutralizing antibodies were observed to reduce collagen accumulation of keloid fibroblasts to a level comparable to the normal fibroblasts or when the uPA activity was increased by culturing keloid fibroblasts in collagen containing matrix gels. Filled block arrows indicate activity levels.

### DETAILED DESCRIPTION OF THE INVENTION

The present invention relates to findings that fibroblasts from abnormally formed scars exhibit an excessive accumulation of collagen, express an elevated activity of plasminogen activator inhibitor-1 (PAI-1), and that decreasing the activity of PAI-1 attenuates the excessive deposit of collagen in the fibroblasts from abnormal scars.

In particular, it is discovered through immunohistochemical studies that although dermal fibroblasts of normally formed scars and abnormally formed scars (for example, keloids) both express urokinase type plasminogen activator (uPA) and PAI-1, fibroblasts from abnormally formed scars have a much higher PAI-1 expression. Long-term three-dimensional fibrin gel cultures reveal that normal fibroblasts express moderate and modulated activity levels of uPA and PAI-1. In contrast, keloid fibroblasts expressed a persistently high level of PAI-1 and a low level of uPA. The elevated PAI-1 activity of the keloid fibroblasts correlate with their elevated collagen accumulation in fibrin gel cultures. Furthermore, it is observed that decreasing PAI-1 activity in fibrin gel cultures with anti-PAI-1-neutralizing antibodies reduces the elevated accumulation in the keloid fibroblasts.

While not wishing to be bound to any theory, these findings suggest that PAI-1 over-expression or elevated activity of PAI-1 is a persistent feature of fibroblasts from abnormally formed scars both in vitro and in vivo. Given that decreasing PAI-1 activity leads to the reduction of excessive deposit of collagen, PAI-1 appears to play a causative role in elevated collagen accumulation of fibroblasts from abnormally formed scars. Accordingly, one aspect of the present invention is directed to methods for preventing or reducing an excessive deposit or accumulation of collagen in fibroblasts of abnormal scars or abnormalities in wound healing comprising the step of reducing the activity of PAI-1.

It is known in the art that proteolytic degradation of fibrin matrix and subsequent substitution of collagen produced by fibroblasts are essential features of a wound healing process, especially at the fibroplasia and remodeling stages. It has also been reported that excessive deposition or over-expression of collagen would cause abnormalities in wound healing process that results in abnormal scars. Tuan & Nichter, The Molecular Basis of Keloid and Hypertrophic Scar Formation, Mol. Med. Today 4: 19-24 (1998). While not wishing to be bound to any theory, it appears that the reduction of PAI-1 activity may prevent abnormalities in a wound healing process that are caused by excessive deposit of collagen. In addition, it appears that decreasing the PAI-1 activity may reverse the pathological course of abnormal scar formation and bring back a wound healing process to its normal course. Accordingly, another aspect of the present invention is directed to methods for preventing and/or reducing an abnormality in wound healing or an abnormal scar that results from excessive deposition of collagen comprising the step of reducing the activity of PAI-1.

As the activity of PAI-1 can be measured by methods known in the art, e.g. a chromogenic assay, a fibrin overlay assay, and a reverse fibrin overlay assay as described herein, the level of PAI-1 activity during a normal course of wound healing or a normal scar formation can be determined and set forth as a standard PAI-1 activity. The standard PAI-1 activity in a normal course of a wound healing process can be used to compare with the level of PAI-1 activity at a wound site undergoing a wound healing process. The standard PAI-1 activity in a normal wound healing process can be established using well known methods or assays to determine PAI-1 activity at a wound site, or methods set forth in Gaffney & Edgell, The international standard for plasminogen activator inhibitor-1 (PAI-1) activity, Thromb. Haemost. 76:80-83 (1996). Since abnormally formed scars caused by excessive deposit of collagen express persistently an elevated level of PAI-1, the elevated level of PAI-1 activity shown at the wound site in a wound healing process may represent a likelihood of excessive accumulation of collagen or a propensity of forming an abnormal scar or an abnormality in the wound healing process. Accordingly, another aspect of the present invention is directed to methods for determining the likelihood of excessive deposit of collagen or the propensity of abnormal scar formation comprising the steps of locating a wound site and measuring the level of PAI-1 activity at the wound site. The methods further comprise the steps of comparing the PAI-1 activity at the wound site with a standard PAI-1 activity in a normal wound healing process and determining a likelihood of forming an abnormal scar.

Plasminogen Activator Inhibitor-1 (PAI-1).

PAI-1 as used herein is a member of the serine protease inhibitor (SERPIN) family and is the major inhibitor to both serine protease urokinase type plasminogen activators (uPA) and tissue type plasminogen activators (tPA). It has been found that PAI-inhibition of plasminogen activators is mediated through a bait peptide bond of PAI-1 protein (amino acid residues between #346 (Arg) and #347 (Met)), which mimics the natural substrate for plasminogen activators, plasminogen.

Both uPA and tPA are enzymes that convert plasminogen into plasmin. Plasmin then participates in the breakdown of other glycoproteins in the extracellular matrix (ECM), the activation of matrix metalloproteinases (MMP), and the release of transforming growth factor TGF-B. Rifkin et al., Plasminogen/plasminogen activator and growth factor activation, Ciba. Found. Symp. 212:105-115 (1997). Accordingly, as the primary regulator of plasminogen activation in vivo, PAI-1 appears to be involved in the extracellular matrix metabolism during a wound healing process. It has been reported that the increased expression of PAI-1 in vivo suppresses the normal fibrinolytic system of the tissues and creates a local prothrombotic state which may result in a pathological deposition of fibrin at the site of tissue injury. Yamamoto & Saito, A Pathological Role of Increased Expression of Plasminogen Activator Inhibitor-1 in Human or Animal Disorders, Int'l. J. Hematol. 68: 371-385 (1998).

The molecular basis for PAI-1 has been well characterized. In particular, DNA sequences coding the full length PAI-1 from humen and animals have been cloned and sequenced. For example, the cDNA sequence and its encoding amino acid sequence of a human PAI-1 are listed in Genbank Accession No. X047444. The cDNA sequence and its encoding amino acid sequence of mouse PAI-1 are listed in GenBank Accession No. M33960. PAI-1 as used in the present invention refers to human PAI-1.

One of the features of PAI-1 is that PAI-1 can spontaneously convert from its active conformation into a latent, inactive conformation which is unable to bind to and inhibit plasminogen activators. Sancho, et al., Conformational studies on plasminogen activator inhibitor (PAI-1) in active, latent, substrate, and cleaved forms, Biochem. 34: 1064 -1069 (1995). It is reported that amino acid residues from #333 (Ser) to #346 (Lys) of PAI-1, also called a reactive center loop, are responsible for PAI-1's inhibitory effect on plasminogen activator. Eitzman et al., Peptide-mediated inactivation of recombinant and platelet plasminogen activator inhibitor-1 in vitro, J. Clin. Invest. 95: 2416-2420 (1995). In the active formation of PAI-1, the reactive center loop (RCL) protrudes from the surface of the protein and exposes the bait peptide bond (Arg³⁴⁶-Met³⁴⁷) to plasminogen activators as a pseudosubstrate. However, in the latent, inactive conformation, the reactive center loop is inserted as a central strand into β-sheet A. Id. In addition, a 14-amino acid peptide (an RCL peptide) corresponding to the PAI-reactive center loop has shown to attenuate PAI-1 function and activity. Id.

Reduction of PAI-1 Activity.

The reduction of PAI-1 can be achieved by a method that reduces, decreases, abrogates, or eliminates the expression, activity or existence of PAI-1. For example, PAI-1 activity can be decreased through the removal of PAI-1 gene or protein. It is reported that PAI-1 knockout mice that are successfully produced appear to be protected against bleomycin-induced pulmonary fibrosis. Hattori et al., Bleomycin-Induced Pulmonary Fibrosis in Fibrinogen-Null Mice, J. Invest. Invest. 106: 1341-1350 (2000). PAI-1 activity can also be reduced through increasing uPA activity by culturing fibroblasts in collagen or fibrin-collagen gels in vitro as described herein.

In one embodiment of the invention, PAI-1 activity is reduced by a PAI-1 inhibitor. A PAI-1 inhibitor is a molecule or macromolecule that inhibits (suppresses or down-regulates) the activity of PAI-1 directly or indirectly.

In a preferred embodiment, PAI-1 inhibitor is a direct PAI-1 inhibitor that interacts with or binds to PAI-1 directly and thereby reduces the activity of PAI-1. In a more preferred embodiment, direct PAI-1 inhibitors include but are not limited to 1) diketopiperazines XR330 and XR334, Bryans et al., Inhibition of plasminogen activator inhibitor-1 activity by two diketopiperazines produced by Streptomyces sp., J. Antibiot. 49 1014-1021 (1996); 2) diketopiperazines XR1853 and XR 5082, Charlton et al., Evaluation of a low molecular weight modulator of human plasminogen activator inhibitor-1 activity, Thromb. Haemost. 75: 808-15 (1996); 3) XR5118 and diketopiperazine-based compounds derived from XR5118, e.g., compounds # 24, 25, 33, 34, 35, 36, 37, and 38, as described in Folkes et al., Synthesis and In Vitro Evaluation of a Series of Diketopiperazine Inhibitors of Plasminogen Activator Inhibitor-1, Bioorg. Medicinal Chem. Lett. 11: 2589-2592 (2001), 4) tetramic acid based compounds and hydroxyquinolinone-based compounds as described in Folkes et al., Design, synthesis, and in vitro evaluation of potent, novel, small molecule inhibitors of plasminogen activator inhibitor-1, Bioorg. Med. Chem. Lett. 12: 1063-1066 (2002), and 5) 11-keto-9(E), 12(E)-octadecadienoic acid, Chikanishi et al., Inhibition of plasminogen activator inhibitor-1 by 11-keto-9(E), 12(E)-octadecadienoic acid, a novel fatty acid produced by Trichoderma sp., J. Antibiot. 52: 797-802 (1999). For low molecular weight chemical compounds that inhibit the activity of PAI-1, see also U.S. Pat. No. 5,902,812, U.S. Pat. No. 5,891,877, and U.S. Pat. No. 5,750,535, which are hereby incorporated by reference in their entirety.

In another more preferred embodiment of the invention, the direct PAI-1 inhibitors include PAI-1 neutralizing antibodies as described herein, and PAI-1 inhibitory monoclonal antibodies including but not limited to murine monoclonal antibodies against human PAI-1 MA-44E4, MA-42A2F6, MA-56A7C10, MA-33B8. See, Verhamme et al, Accelerated conversion of human plasminogen activator inhibitor-1 to its latent form by antibody binding, J. Biol. Chem., 274: 17522-17517 (1999); Bijnens et al, The distal hinge of the reactive site loop and its proximity: A target to modulate plasminogen activator inhibitor-1 activity, J. Biol. Chem., 276: 44912-44918 (2001). Since the reactive center loop (Ser³³³-Lys³⁴⁶ of PAI-1) is protruded from the surface of PAI-1 structure and presents a bait peptide bond (Aug³⁴⁶-Met³⁴⁷) to plasminogen activators, polyclonal or monoclonal antibodies against the reactive center loop is contemplated to be a direct PAI-1 inhibitor. PAI-1 neutralizing or inhibitory antibodies may bind to PAI-1 and block its activity through inhibiting its interaction with plasminogen activators. Alternatively, PAI-1 neutralizing or inhibitory antibodies may suppress PAI-1 activity by accelerating the conversion of an active conformation of PAI-1 into a latent, inactive form. See, Verhamme, supra.

In another embodiment of the present invention, a PAI-1 inhibitor is an indirect PAI-1 inhibitor which is a molecule or macromolecule that inhibits (suppresses or down-regulates) the activity of PAI-1 indirectly. For example, at the cellular and molecular level, an indirect PAI-1 inhibitor can be a factor or compound that specifically inhibits the transcription or expression of the PAI-1 gene, an antisense oligonucleotide complementary to PAI-1 sequence that blocks the expression of PAI-1, antisense oligonucleotides, a polynucleotide construct that induces RNA interference for the degradation of PAI-1 mRNA, or dicers that produce siRNAs which in turn degrade mRNA of PAI-1, molecules that compete with the PAI-1 in enzymatic reactions with plasminogen activators. It is known in the art that the expression of PAI-1 can be enhanced by factors such as endotoxin, thrombin, TNF-alpha, TGF-β, interleukin-1, insulin, dexamethasone, PDGF, EGF, lipoprotein, and angiotensin II. Accordingly, an indirect PAI-1 inhibitor may be an inhibitor against the factors thereof which indirectly reduce the expression of PAI-1.

It is more preferred that an indirect PAI-inhibitor be a compound that suppresses the expression of PAI-1. It is known in the art that indirect PAI-1 inhibitors that suppress or attenuate the expression of PAI-1 include but are not limited to angiotensin-converting enzyme inhibitors (e.g., Fosinopril, Imidapril, Captopril, Enalapril); angiotensin II receptor antagonists (LI 58,809, Eprosartan); Troglitazone; Vitamin C; Vitamin E; Perindorpril; Mifepristone (RU486); and Spironolactone. See, Eitzman et al., Peptide-mediated inactivation of recombinant and platelet plasminogen activator inhibitor-1 in vitro, J. Clin. Invest. 95:2416-2420 (1995); Pawlowska et al., Natriuretic peptides reduce plasminogen activator inhibitor-1 expression in human endothelial cells, Cell Biol. Lett. 7:1153-1157 (2002); Mitsui et al., Imidapril, an angiotensin-converting enzyme inhibitor, inhibits thrombosis via reduction in aortic plasminogen activator inhibitor type-1 levels in spontaneously hypertensive rats, Biol. Pharm. Bull. 22:863-865 (1999); Brown et al., Aldosterone modulates plasminogen activator inhibitor-1 and glomerulosclerosis in vivo, Kidney Int. 58:1219-1227 (2000); Wong et al., Gene expression in rats with renal disease treated with the angiotensin II receptor antagonist, Eprosartan, Physiol. Genomics 4:35-42 (2000); Papp et al., Biological mechanisms underlying the clinical effects of mifepristone (RU 486) on the endometrium, Early Pregnancy 4:230-239 (2000); Oikawa et al., Modulation of plasminogen activator inhibitor-1 in vivo: a new mechanism for the anti-fibrotic effect of renin-angiotensin inhibition, Kidney Int. 51:164-172 (1997); Fogari et al., Losartan and perindopril effects on plasma plasminogen activator inhibitor-1 and fibrinogen in hypertensive type 2 diabetic patients, Am. J. Hypertens. 15:316-320 (2002); Pahor et al., Fosinopril versus amlogipine comparative treatments study: a randomized trial to assess effects on plasminogen activator inhibitor-1, Circulation 105:457-461 (2002); Orge et al., Vitamins C and E attenuate plasminogen activator inhibitor-1 (PAI-1) expression in a hypercholesterolemic porcine model of angioplasty, Cardiovasc. Res. 49:484-492 (2001); Gottschling-Zeller et al., Troglitazone reduces plasminogen activator inhibitor-1expression and secretion in cultured human adipocytes, Diabetologia 43:377-383 (2000); Katoh et al., Angiotensin-converting enzyme inhibitor prevents plasminogen activator inhibitor-1 expression in a rat model with cardiovascular remodeling induced by chronic inhibition of nitric oxide synthesis, J. Mol. Cell Cardiol. 32:73-83 (2000).

In another more preferred embodiment, indirect PAI-inhibitors are peptides that interfere with the reaction between PAI-1 and plasminogen activators and therefore indirectly reduce PAI-1 activity. For example, a peptide (an RCL peptide) containing the sequence of the reactive center loop of PAI-1 is known to inhibit PAI-1 activity. Verhamme, supra.

In determining whether a molecule or a macromolecule is a PAI-1 inhibitor, a chromogenic assay known to one of ordinary skills in the art is often conducted to measure PAI-1 activity in the presence of the molecule or the macromolecule. In the chromogenic assay, the molecule is first mixed to a solution containing PAI-1 or a cell culture containing cells secreting PAI-1. A fixed amount of tissue plasminogen activator is then added to the resultant mixture and allowed to react with PAI-1. The residue tPA is measured by adding to the reaction a mixture of Glu-plasminogen, poly D-lysine and chromogenic substrate at neutral pH. The residue tPA activity catalyzes the conversion of plasminogen to plasmin which further hydrolyzes the chromogenic substrate. The degree of color revealed proportionally correlates to the amount of tPA which in turn represents the inhibitory nature and effectiveness of the molecule. The chromogenic assay is detailed in Wysocki et al, Temporal Expression of Urokinase Plasminogen Activator, Plasminogen Activator Inhibitor and Gelatinase-A in Chronic Wound Fluid Switches from a Chronic to Acute Wound Profile with Progression to Healing, Wound Repair Regen. 7: 154-165 (1999). Additionally, whether a molecule suppresses the expression of PAI-1 can also be determined by Fibrin Overlay Assay, Reverse Fibrin Overlay Assay, Enzyme-Linked Immunosorbent Assay (ELISA), all of which are well known in the art and/or described herein.

To examine the effectiveness of a reduction of excessive collagen accumulation caused by a PAI-inhibitor, an in vitro three dimensional fibrin matrix gel culture system is used. The 3-D fibrin matrix gel culture system is an in vitro fibroplasia model that is established to study the interplay between cells and extracellular matrix during a wound healing process. Tuan et al., In vitro Fibroplasia: Matrix Contraction, Cell Growth and Collagen Production of Fibroblasts Cultured in 3-Dimensional Fibrin Matrix, Exp. Cell Res. 223: 127-134 (1996). The 3-D fibrin matrix gel culture system presents key features of fibroplasia. In particular, the system mimics cell proliferation, fibrin reorganization and degradation, and collagen synthesis and deposition in wound healing. Therefore, the system effectively represents the in vivo process of fibroplasia. The presence of a PAI-1 inhibitor in the 3-D fibrin matrix gel culture system causes a reduction of the activity of PAI-1 and subsequently a reduction of collagen synthesis. The level of collagen synthesis can be determined using a method known in the art. Tuan et al., In vitro Fibroplasia: Matrix Contraction, Cell Growth and Collagen Production of Fibroblasts Cultured in 3-Dimensional Fibrin Matrix, Exp. Cell Res. 223: 127-134 (1996).

Since the activity of PAI-1 can be measured using the chromogenic assay, the level of PAI-1 activity during the course of normal scar formation can thus be determined and set forth as a standard PAI-1 activity in comparison with the activity of PAI-1 in the course of abnormal scar formation. The propensity of abnormal scar formation can therefore be determined at each stage of scar formation and a PAI-1 inhibitor can be administered in a pertinent therapeutically effective amount to prevent abnormal scar formation or reduce the likelihood of forming an abnormal scar.

Abnormalities in Wound Healing

As mentioned, one aspect of the invention is directed to methods of averting abnormal scar formation or treating abnormalities in wound healing caused by an excessive deposit of collagen comprising the step of reducing PAI-1 activity. The term “wound” as used herein is exemplified by but not limited to injury, damage or trauma to at least the membranous or epithelial layers of the inner and outer surface of a body or the body's tissues or organs such as skin, lung, kidney, liver, heart, gastrointestinal tract, bone, tendon, eye, or nerve. A wound can be caused by trauma, surgery, incision, infection, burn, abrasion, puncture, strike, blister, pollutants, or toxins. Once a wound occurs, the wounded area or the wound site usually undergoes a wound healing process in order to repair the injured tissue or organ. A normal wound healing process would bring the tissue or organ at the wound site back to as much as possible its unwounded condition. However, a wound healing process is a dedicated process involved in many stages and influenced by many factors as described. Any aberrations may disturb the process and lead to an abnormality or an abnormal scar formation as a deviate from a normal wound healing.

The terms “abnormal scar”, “abnormal scar formation”, “abnormality in wound healing” or “disorder in wound healing” or “wound healing disorder” as used herein refers to deviations from a normal wound healing process that are caused by excessive deposit or accumulation of collagen. The abnormal scars or the abnormalities in wound healing includes but are not limited to fibrosis, fibromatosis, keloidosis, adhesions (e.g. surgical adhesions), hypertrophic scars, fibrocystic conditions, and joint stiffness. Abnormal scars or abnormalities in wound healing can also be categorized into various conditions based on the type of tissue in which a wound occurs. Abnormal scar formation in skin may lead to, for example, keloid, hypertrophic scar, contracture, or scleroderma. Abnormalities in wound healing in the gastrointestinal tract may lead to, for example, stricture, adhesion, or chronic pancreatitis. Abnormalities in wound healing may cause, for example, glomerulonephritis in kidneys, retrolenthal fybroplasis in eyes, cirrhosis and biliary atresia in livers, intersticial fibrosis or bronchoplumonary dysplasia in lungs, and rheumatic disease or ventricular aneurysm in hearts. See, Sabiston Textbook of Surgery: The Biological Basis of Modem Surgical Practice, Chapter 12 (16^{th} Ed., 2001).

It is preferred that wound healing disorders or abnormal scars associated with skin include, but are not limited to, a hypertrophic scar, a keloid, a skin disfiguring problem including acne, wrinkling, cellulite formation and neoplastic fibrosis, a Duypuytren's disease, a Peyronie's disease, and other cutaneous or internal wounds or lesions in skin. It is more preferred that the abnormal scar formation include a hypertrophic scar, a keloid, and a skin disfiguring problem. A keloid results from excessive deposition of scar tissue that proliferates beyond the boundary of the original wound. A hypertrophic scar forms when the excessive deposition of scar tissue is confined to the margin of the original wound. It is known in the art that in both keloid and hypertrophic scars, excessive scarring is caused by pathologically over-expression and accumulation of collagen. Haverstock, Hypertrophic Scars and Keloids, Clin. Podiatr. Med. Surg. 18: 147-159 (2001).

It is further preferred that a wound healing disorder is fibrosis. Fibrosis shows excessive collagen accumulation and impairs the function of a tissue or organ when wound sites in tissues are replaced with abnormal scars. Examples of fibrosis include but are not limited to the formation of scar tissue following a heart attack which impair the ability of the heart to pump, abnormal scarring in kidney from diabetes which leads to a progressive loss of kidney function, and fibrous adhesions between organs after surgery which cause contracture and pain. Major organ or tissue based fibrosis includes but is not limited to kidney fibrosis caused by diabetes or hypertension, liver fibrosis caused by alcohol or viral hepatitis, pulmonary fibrosis, cardiac fibrosis, macular degeneration, and retinal and vitreal retinopathy.

The term “excessive accumulation of collagen”, “excessive deposit of collagen”, or “over-expression of collagen” as used herein refer to an elevated level of collagen at a wound site or in a scar which is higher than the normal level of collagen at a wound site undergoing a normal healing process or in a normally formed scar. It is prefered that the elevated level of collagen is about at least 20% higher than the normal level. It is more preferred that the elevated level of collagen is about at least 30% higher. The level of collagen accumulation can be determined using in vivo assays and in vitro assays. In the in vivo assays, the amount of collagen present at a wound site or in a scar is measured by morphological assessment or biochemical assessment using punch biopsy as well known in the art. In the in vitro assays, fibroblasts from a wound site are collected and placed into a in vitro three-dimensional fibrin matrix culture system. Fibroblasts (control fibroblasts) from a normal scar or a normal tissue are used as a control. Newly sysnthesized collagen from these fibroblasts is purified and measured by using labeled amino acids. See, Tuan et al., In vitro fibroplasia: matrix contraction, cell growth, and collagen production of fibroblasts cultured in fibrin gels, Exp. Cell. Res. 223: 127-134 (1996). If the level of newly synthesized collagen is higher, preferably about at least 20% higher, more preferably about at least 30% higher, than that of control fibroblasts, the wound site or the scar can be deemed to have an excessive deposit or accumulation of collagen.

Administration of PAI-1 Inhibitors

One embodiment of the invention is directed to methods for preventing abnormal scar formation or treating abnormal scars by administering a PAI-1 inhibitor composition to a subject inflicted with a wound. The PAI-1 inhibitor composition is either a PAI-1 inhibitor by itself or a PAI-1 inhibitor medicament which comprises a PAI-1 inhibitor and a pharmaceutically acceptable carrier.

The PAI-1 inhibitor composition can be administered to a subject by any administration route known in the art, including without limitation, oral, enteral, buccal, nasal, topical, rectal, vaginal, aerosol, transmucosal, epidermal, transdermal, ophthalmic, pulmonary, and/or parenteral administration. The epidermal or topical administration refers to the delivery of the PAI-1 inhibitor directly onto a wound site. The conjunctival administration refers to the delivery of the PAI-1 inhibitor across the corneal and conjunctival surface into the eye and/or to the rest of the body and the wound site. The nasal administration refers to the delivery of the PAI-1 inhibitor across the nasal mucous epithelium and into the peripheral circulation. The buccal administration refers to the delivery across the buccal or lingual epithelia into the peripheral circulation. The oral administration refers to the delivery of the PAI-1 inhibitor through the buccal epithelia but predominantly swallowed and absorbed in the stomach and alimentary tract. The rectal administration refers the delivery of the PAI-1 inhibitor via the lower alimentary tract mucosal membranes into the peripheral circulation. The vaginal administration refers to the delivery of the PAI-1 inhibitor through vaginal mucous membrane into the peripheral circulation. The peripheral circulation carries the PAI-1 inhibitor to the wound site. A parenteral administration refers to an administration route that typically relates to injection which includes but is not limited to intravenous, intramuscular, intraarterial, intrathecal, intracapsular, intraorbital, intra cardiac, intradermal, intraperitoneal, transtracheal, subcutaneous, subcuticular, intraarticular, subcapsular, subarachnoid, intraspinal, and/or intrasternal injection and/or infusion.

The term “pharmaceutically acceptable carrier” as used herein means a pharmaceutically-acceptable material, composition or vehicle, such as a liquid or solid filler, diluent, excipient, solvent or encapsulating material, involved in carrying or transporting a PAI-1 inhibitor from one tissue, organ, or portion of the body, to another tissue, organ, or portion of the body. Each carrier must be “pharmaceutically acceptable” in the sense of being compatible with the other ingredients, e.g., a PAI-1 inhibitor, of the formulation and suitable for use in contact with the tissue or organ of humans and animals without excessive toxicity, irritation, allergic response, immunogenicity, or other problems or complications, commensurate with a reasonable benefit/risk ratio. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; ( 1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Typically, a PAI-1 inhibitor composition is given to a subject in the form of formulations or preparations suitable for each administration route. The formulations useful in the methods of the present invention include one or more PAI-1 inhibitors, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of a PAI-1 inhibitor which can be combined with a carrier material to produce a pharmaceutically effective dose will generally be that amount of a PAI-1 inhibitor which produces a therapeutic effect. Generally, out of one hundred per cent, this amount will range from about 1 per cent to about ninety-nine percent of the PAI-1 inhibitor, preferably from about 5 per cent to about 70 per cent.

Methods of preparing these formulations or compositions include the step of bringing into association a PAI-1 inhibitor with one or more pharmaceutically acceptable carriers and, optionally, one or more accessory ingredients. In general, the formulations are prepared by uniformly and intimately bringing into association a PAI-1 inhibitor with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non- aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a PAI-1 inhibitor as an active ingredient. A compound may also be administered as a bolus, electuary, or paste.

In solid dosage forms for oral administration (e. g., capsules, tablets, pills, dragees, powders, granules and the like), the PAI-1 inhibitor is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of a PAI-1 inhibitor therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the PAI-1 inhibitor(s) only, or preferentially, in a certain portion of the gastrointestinal tract, optionally, in a delayed manner. Examples of embedding compositions which can be used include polymeric substances and waxes. The PAI-1 inhibitor can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the PAI-1 inhibitor, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcoho, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the PAI-1 inhibitor, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more PAI-1 inhibitors with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Formulations which are suitable for vaginal administration also include pessaries, tampons, creams, gels, pastes, foams or spray formulations containing such carriers as are known in the art to be appropriate.

Formulations for the topical or transdermal or epidermal administration of a PAI-1 inhibitor composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. The ointments, pastes, creams and gels may contain, in addition to the PAI-1 inhibitor composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the PAI-1 inhibitor composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

PAI-1 inhibitor compositions can be alternatively administered by aerosol. This is accomplished by preparing an aqueous aerosol, liposomal preparation or solid particles containing the PAI-1 inhibitors. A nonaqueous (e. g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches can also be used to deliver PAI-1 inhibitor compositions to an abnormal scar. Such formulations can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Formulations suitable for parenteral administration comprise a PAI-1 inhibitor in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e. g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a PAI-1 inhibitor, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered formulation is accomplished by dissolving or suspending the PAI-1 inhibitor composition in an oil vehicle.

Injectable depot forms are made by forming microencapsule matrices of a PAI-1 inhibitor or in biodegradable polymers such as polylactide-polyglycolide. Depending on the ratio of the PAI-1 inhibitor to polymer, and the nature of the particular polymer employed, the rate of drug release can be controlled. Examples of other biodegradable polymers include poly (orthoesters) and poly (anhydrides). Depot injectable formulations are also prepared by entrapping the PAI-inhibitor in liposomes or microemulsions which are compatible with body tissue.

In a preferred embodiment of the invention, a PAI-1 inhibitor composition is delivered to a wound site in a therapeutically effective dose. The term “pharmaceutically effective dose” as used herein refers to the amount of a PAI-1 inhibitor, a PAI-1 inhibitor composition, or a PAI-1 inhibitor medicament, which is effective for producing a desired therapeutic effect, or which is reflected by reducing an excessive accumulation or expression of collagen in a wound healing that would lead to the formation of an abnormal scar, or bringing down the level of collagen accumulation in an abnormality in would healing to that of normal wound healing, or observing a normal scar formation at a wound site with a propensity to form an abnormal scar were the PAI-1 inhibitor not to be administered, or observing the remission of an abnormally formed scar. As is known in the art of pharmacology, the precise amount of the pharmaceutically effective dose of a PAI-inhibitor that will yield the most effective results in terms of efficacy of treatment in a given patient will depend upon, for example, the activity, the particular nature, pharmacokinetics, pharmacodynamics, and bioavailability of a particular PAI-1 inhibitor, physiological condition of the subject (including race, age, sex, weight, diet, disease type and stage, general physical condition, responsiveness to a given dosage and type of medication), the nature of pharmaceutically acceptable carriers in a formulation, the route and frequency of administration being used, and the severity or propensity of a wound or an abnormal scar formation, to name a few. However, the above guidelines can be used as the basis for fine-tuning the treatment, e. g., determining the optimum dose of administration, which will require no more than routine experimentation consisting of monitoring the subject and adjusting the dosage. Remington: The Science and Practice of Pharmacy (Gennaro ed. 20^{th} edition, Williams & Wilkins PA, USA) (2000).

Having generally described the present invention, the same will be better understood by reference to certain specific examples, which are set forth herein for the purpose of illustration.

### EXAMPLES

Materials and Methods

Cell Isolation: Fibroblasts were established from donors of human normal skin, scar, and keloid using the explant method. The protocol for skin and scar collections was approved by both Children's Hospital Los Angeles and Charles R. Drew University of Medicine and Science. The raised core region of keloid scars was used for fibroblast isolation. Fibroblasts were grown in Dulbecco's Modified Eagle's Medium (DMEM) (Life Technologies, Inc., Grand Island, N.Y.) containing 100 U/ml penicillin, 100 μg/ml streptomycin, and 10% fetal bovine serum (Life Technologies, Inc.). Cultures were incubated in a humidified incubator in an atmosphere of 5% CO₂ and 95% air. Fibroblasts were harvested from cultures using 0.25% trypsin containing 0.05% ethylenediamine tetraacetic acid in Hanks' solution (Life Technologies, Inc.) and passaged once a week. Early passages (2-10) of fibroblasts were used in the experiments. Cell passage is defined as weekly expansion of cells from primary cultures. The source of each strain of fibroblasts used in the present invention is listed in Table 1. These specimens represented a research effort in sample procurement throughout an 8-year period. Each experiment presented in the invention was conducted and compared between multiple strains of normal and keloid fibroblasts in pairs matching donor age and anatomical site whenever possible.

Preparation of fibrin eels: Human fibrinogen (Calbiochem, San Diego, Calif.) was used for the preparation of fibrin gels. Fibrinogen was reconstituted in distilled H₂O, adjusted to 10 mg/ml, and stored at −20° C. The clotability of fibrinogen was determined by mixing 1-5 mg/ml fibrinogen with 1-2 units/ml of human thrombin and incubating for 30 min at 37° C. The clots that formed were detached from test tube walls. Tubes were centrifuged at 12,000 g for 15 min to pellet the clot and collect soluble fibrinogen. The soluble non-clotable fibrinogen remaining in the supernatant was determined by protein concentration at OD₂₈₀. All fibrinogen used was 95 to 98% clotable.

The method for fibrin gel preparation has been described in a previous publication. Tuan & Grinnell, Fibronectin and fibrinolysis are not required for fibrin gel contraction by human skin fibroblasts, J. Cell Physiol. 140: 577-583 (1989). Briefly, human skin fibroblasts in DMEM were added to a fibrinogen solution at 24° C. Final concentrations of fibrinogen and fibroblasts were 2.5 mg/ml and 0.5×10⁶ cells/ml, respectively. Aliquots (180 ml) of the fibroblast/fibrinogen mixtures were placed in wells of 24-well tissue culture plates (Costar, Cambridge, Mass.) with 1 unit of thrombin per sample. Each aliquot occupied an area outlined by a 16-mm-diameter circular score within the well. The preparations were incubated at 37° C. for 1 hour in a humidified incubator containing 5% CO₂ to ensure polymerization of fibrin. At the end of the incubation period, 1.0 ml of DMEM containing 10% FCS was added to each well in order to cover the gel.

Samples selected for uPA and PAI-1 studies were first thoroughly rinsed (5 times) with DMEM and incubated in DMEM for an additional 24 hrs. Conditioned culture media were collected and subjected to fibrin overlay and reverse fibrin overlay assays.

Preparation of collagen gels: Collagen gels were prepared according to the method previously described by Tuan et al., Dermal fibroblasts activate keratinocyte outgrowth on collagen gels, J. Cell. Sci. 107: 2285-2289 (1994). Vitrogen (Cohesion Technologies, Inc., Palo Alto, Calif.), a preparation of predominantly type I collagen was used. Briefly, the collagen was adjusted to physiologic ionic strength and pH with 10× minimum essential medium (MEM) (Sigma Chemical Company) and 0.1 N NaOH at 4° C. The final collagen concentration was 1.5 mg/ml. Fibroblasts were incorporated into the reconstituted collagen at a final concentration of 0.5×10⁶ cells/ml. Samples of the collagen/fibroblast suspension were dispensed into 24-well culture plates. Each 180-μl aliquot was contained within a circle of 16-mm diameter scored onto the base of the well. The culture plates were then placed in an incubator at 37° C. with 5% CO₂ for 45 minutes to allow collagen to polymerize.

Fibrin and collagen mixture gels: Gels were prepared by mixing fibrinogen and collagen in different ratios (fibrin: collagen; 100%:0%; 50%:50%; 0%:100%). Fibroblasts were incorporated into the matrix at a final density of 0.5×10⁶ cells/ml. Aliquots (180-μl) of gel-fibroblast mixtures were placed in wells of 24-well tissue culture plates with 1 unit of thrombin per sample following a similar format described above.

Fibrin overlay and reverse overlay: Briefly, aliquots (25 μl) of serum-free conditioned culture media were subjected to electrophoresis using a 10% polyacrylamide gel containing 0.1% sodium dodecyl sulfate (SDS, Sigma). The gel was washed for 1 hour at room temperature in 2.5% Triton X-100 to remove SDS. After a brief rinse in distilled water, the gel was placed on an indicator gel layer (fibrin overlay assay for Plasminogen Activator (PA) detection) that contained 1% low-temperature gelling agarose, human plasminogen (9 μg/ml, Sigma, St. Louis, Mo.), thrombin (0.7 U/ml, Sigma), and fibrinogen (2 mg/ml). To detect PAI, SDS-polyacrylamide gels were washed in 2.5% Triton X-100 for 1 hour at room temperature and placed on top of a substrate gel similar to the indicator gel (above) with the addition of uPA (0.2 U/ml, Sigma) (reverse fibrin overlay assay). Both preparations were placed in a humidified chamber at 37° C. Activity of PA appeared as clear zones in the opaque fibrin indicator layer indicating fibrinolysis. Activity of PAI appeared as opaque zones in a cleared reversed overlay substrate layer indicating inhibition of fibrinolysis. The results were photographed.

Chromogenic substrate assay: A two-stage, indirect enzymatic assay, Spectrolyse (pL) PAI (American Diagnostica # 101201), was used for the quantitative determination of PAI-1 activity in plasma. In stage one, a fixed amount of tissue plasminogen activator (tPA) was added to the sample and allowed to react with PAI-1 present. The sample was then acidified to destroy α-2-anti-plasmin and other potential plasmin inhibitors that would otherwise interfere with the tPA assay. In stage two, the residual tPA activity was measured by adding the sample to a mixture of Glu-plasminogen, poly D-lysine and a chromogenic substrate at neutral pH. The residual tPA activity in the sample catalyzed the conversion of plasminogen to plasmin, which in turn hydrolyzes the chromogenic substrate. The amount of color developed is proportional to the amount of tPA activity in the sample. Poly D-lysine is a stimulator of the tPA catalyzed conversion of plasminogen to plasmin. The PAI content of the sample is then identified as the difference between the amount of tPA added and the amount of tPA recovered. One U of PAI activity (U) is defined as the amount of PAI that inhibits one IU of a human single chain tPA as calibrated against the International Standard for tPA lot 86/670 distributed by NIBSAC, Holly Hill, London, England.

Collagen synthesis, purification's, and phenotype analysis: [³H]proline was used to label newly synthesized collagen by fibroblasts. Samples in triplicates were labeled for 48 hours with L-(5-[³H]proline) (50 μCi/ml) (Amersham, Arlington Heights, Ill.) in DMEM-10% FCS supplemented with β-aminoproprionitrile (62.5 μg/ml). At the end of labeling, all samples were adjusted to 0.5 M acetic acid and treated with 1 mg/ml pepsin (PM grade, Worthington, Freehold, N.J.) for 24 hour at 4° C. to digest proteins other than intact collagen. Pepsin was inactivated by adding Tris to 50 mM and titration to pH 7.4. Collagen was purified by sequential neutral salt and acid salt precipitation as described previously. Tuan et al., In vitro fibroplasia: matrix contraction, cell growth and collagen production of fibroblasts cultured in fibrin gels, Exp. Cell. Res. 223: 127-134 (1996). The final collagen pellet was rinsed in 50 mM Tris and 40% ethanol and dissolved in 0.5 M acetic acid. Samples were subjected to SOS polyacrylamide gel electrophoresis and followed by fluorography. Samples designated for cell count were treated with trypsin and collagenase, and viable cell numbers were estimated using a hemocytometer in the presence of Trypan Blue. Purified collagen was expressed as cpm/cell. Data presented were an average of three replicate samples. Statistical differences between and within groups were assessed using one-way analysis of variance.

Northern blots: Standard Northern blot analysis was used to study RNA expression. Sambrook et al., Molecular Cloning. A Laboratory Manual. (New York Cold Spring Harbor Laboratory Press, 1989). Briefly, RNA samples were extracted using guanidinium thiocyanate and separated by centrifugation through cesium chloride. Total RNA (20 μg/lane) was separated by electrophoresis, transferred to nylon filters, and baked at 80° C. under vacuum for 2 h. After prehybridization, the radioactive-labeled DNA probes were hybridized to filters for 20 h at 40° C., washed, and visualized by exposure to x-ray film at −70° C. The cDNA probes were labeled according to the method as described in Feinberg & Vogelstein, A technique for radiolabeling DNA restriction endonuclease fragments to high specific activity, Addendum, Anal. Biochem. 137: 266-267 (1984). All samples were standardized to the level of expression of β-actin in each cell strain. Specific human cDNA probes for uPA nucleotides 623-1039 and PAI-1 cDNA (full length) were used as hybridization probes. Laug et al., Complex expression of the genes coding for plasminogen activators and their inhibitors in HeLa-smooth muscle cell hybrids, Cell Growth Differ. 3: 191-197 (1992).

Immunohistochemistry: Freshly collected skin and scar samples were rinsed in ice-cold PBS and fixed in 4% paraformaldehyde (Sigma, pH 7.5) at 4° C. for 24 hours. Samples were treated with 70% ethanol for 24 hours before dehydration. Following dehydration, samples were embedded in paraffin (60° C.), and 5 μm thickness sections were prepared using a microtome. Sections were re-hydrated and treated with H₂O₂. To minimize non-specific binding, sections were first treated with 1.5% BSA/PBS for 30 min at room temperature. Mouse monoclonal antibody against human uPA at 1:50 dilution (#3698 and #394, American Diagnostica Inc., Greenwich, Conn.) and murine monoclonal antibody against human PAI-1 at 1:25 dilution (#3785, American Diagnostica Inc.) were used to detect uPA and PAI-1, respectively. After primary antibody treatment, sections were washed 3 times with PBS and incubated with horse radish peroxidase conjugated secondary antibodies (Amersham Pharmacia Biotech Limited, Buckinghamshire, England) for 50 min. After thorough rinsing with PBS, sections were treated with 3,3′-diaminobenzidine (DAB, Sigma) to reveal antibody-antigen reaction. Sections were also stained lightly with Hematoxylin for nuclear staining.

Results

PAI-1 Expression is Increased in Fibroblasts of Keloid Lesions

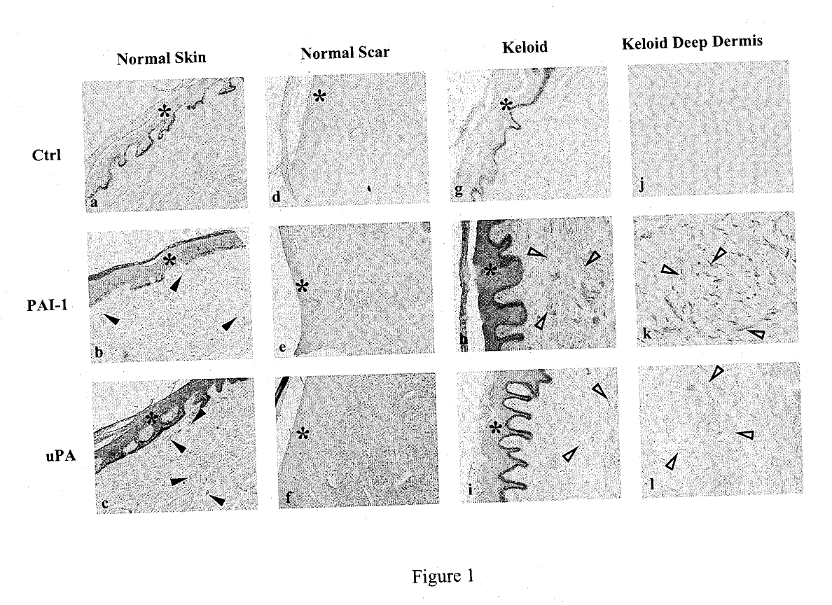

Keloid fibroblasts exhibit elevated PAI-1 expression in culture. Tuan et al., Elevated levels of plasminogen activator inhibitor-1 may account for the altered fibrinolysis by keloid fibroblasts, J. Invest. Dermatol. 106: 1007-1011 (1996). To examine if PAI-1 over-expression also occurs in vivo, protein expressions of both PAI-1 and uPA were studied in keloid lesions (n=5) using antibodies against PAI-1 or uPA in immunohistochemistry. The results were compared with normal skin (n=3) and normal scar (n=3) samples. Keloids are characterized by their overabundance of collagen deposition in the dermis, thus the present study also includes the deep dermal region of keloid lesions (FIG. 1, Keloid Deep Dennis: j, k, and 1). Besides collagen, fibroblasts and blood vessels of various sizes were the major visible structural components in the dermis (FIG. 1). In normal skin, staining of PAI-1 and uPA was localized to the blood vessels (FIG. 1b and 1c). In normal scars and keloids, although both blood vessels and fibroblasts stained positive for uPA and PAI-1, the intensity of their staining was quite different. PAI-1 staining appeared much stronger in keloid fibroblasts than in normal scar fibroblasts (FIGS. 1h & 1k vs. 1e); and uPA staining was stronger in normal scar fibroblasts than in keloid fibroblasts (FIGS. 1f vs. 1i & 1L). The high level of PAI-1 staining was observed in 4 out of 5 (80%) keloid specimens. The epidermis was also positive for uPA and PAI-1 (FIG. 1 “*”) and again the epidermis of keloids showed a stronger PAI-1 staining than that of normal skin or normal scar (FIGS. 1h vs. 1e & 1b). Keloid and normal skin samples were collected from African-American patients where melanocytes in the basal layer of epidermis appeared dark brown in immunohistochemistry. Staining was negative in all control groups (FIG. 1, Ctrl: a, d, g, and j).

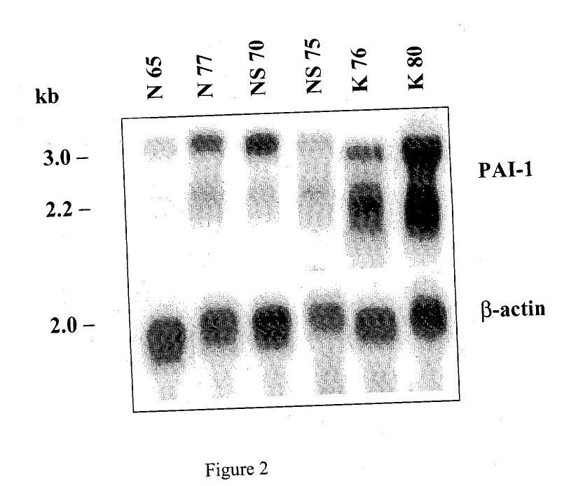

To determine if PAI-1 over-expression also occurred at the mRNA level, skin fibroblasts were isolated from normal skin, normal scar, and keloid samples and analyzed using Northern Blot technique. Results showed that PAI-1 had two RNA messages, 3.0 kb and 2.2 kb, respectively (FIG. 2). The 2.2 kb PAI-1 mRNA in keloid fibroblasts was consistently higher than both normal skin and normal scar fibroblasts. Therefore, PAI-1 over-expression is a consistent feature of keloid fibroblasts both in vitro and in vivo.

Keloid Fibroblasts Exhibit Elevated Collagen Accumulation and Persistently High PAI-1 Activity in Long Term Fibrin Gel Cultures

To examine if PAI-1 over-expression in keloid fibroblasts correlated with their collagen overproduction, PA/PAI and collagen production were studied over a 2-week period employing the in vitro fibroplasia model. Tuan et al., In vitro fibroplasia: matrix contraction cell Growth, and collagen production of fibroblasts cultured in fibrin gels, Exp. Cell Res. 223: 127-134 (1996). Experiments were conducted each time using one keloid and one normal strain of fibroblasts, and a minimum of 6 keloid and 6 normal strains of fibroblasts were examined.

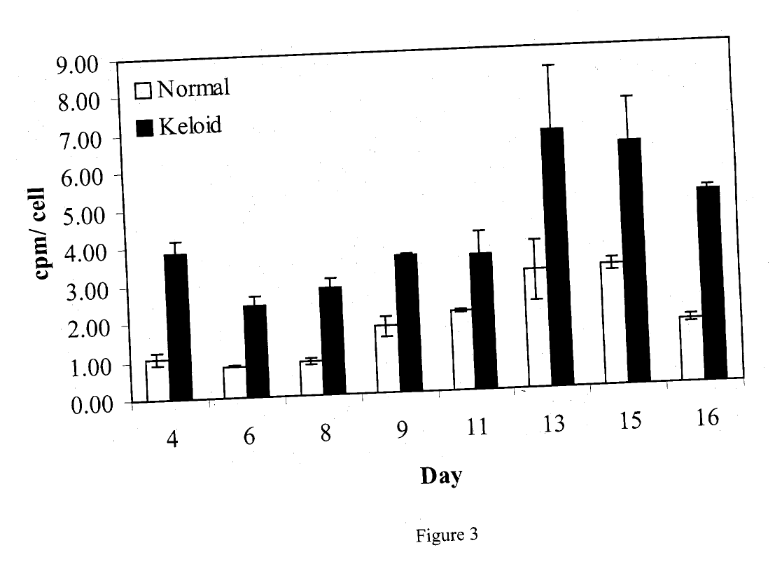

Total collagen produced by fibroblasts was purified as described herein. In fibrin gels, keloid fibroblasts grew at a similar rate as normal fibroblasts. Although a small amount of type III (γ) and type V (ν) collagen was detected, type I collagen (α₁ and α₂) was the predominant collagen made by keloid and normal fibroblasts. Results of a typical experiment are shown in FIG. 3. The quantity of total collagen was normalized to the cell number and expressed as cpm/cell. In the 2-week study, general patterns of collagen accumulation were highly reproducible among different strains of each cell type. For normal fibroblasts, collagen accumulation increased gradually in the first 10 days. It peaked around 13 to 15 days, and decreased at the end of the culture period (16th day) (FIG. 3, Normal). On the other hand, Keloid fibroblasts showed a similar increase in collagen accumulation. However, the level was persistently 2- to 3-fold higher than that of the normal fibroblasts (FIG. 3, Keloid). The elevated level of collagen higher than the level of collagen in normal fibroblasts refers to an excessive accumulation of collagen.

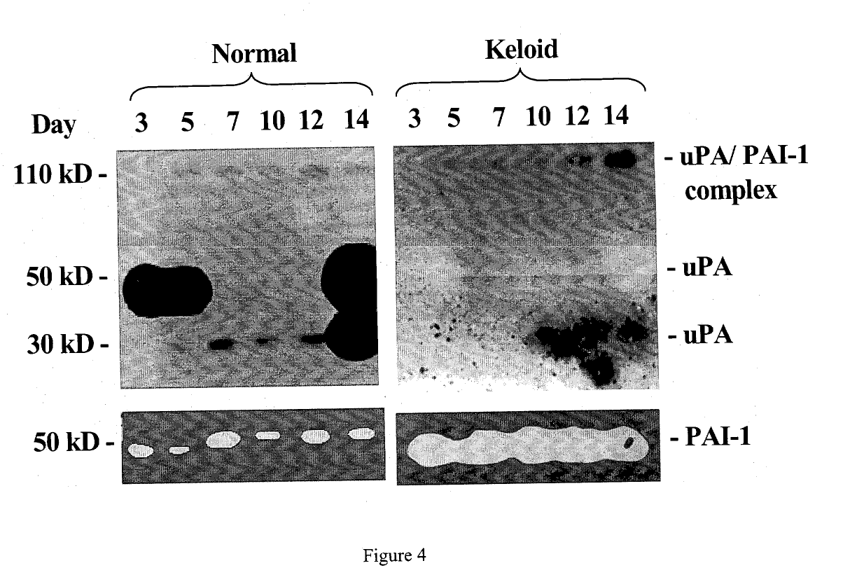

To detect μPA and PAI and their activities, conditioned media were collected from cultures at designated time points and subjected to fibrin overlay and reverse overlay assays. A minimum of 4 strains each of normal and keloid fibroblasts were examined. The result of a typical experiment is shown in FIG. 4. Fibrin overlay assay revealed that normal fibroblasts expressed both the two-chain form (50 kD) and the catalytic fragment single chain form (30 kD) of uPA (FIG. 4, Normal: upper panel). The 50 kD uPA was expressed in early culture periods (3 to 5 days) and reappeared in late culture periods (after 12 days). The 30 kD uPA was expressed in a low level throughout most of the culture period and increased to a high level in the later culture period (FIG. 4, Normal: upper panel). In contrast, keloid fibroblasts exhibited a moderate level of 30 kD uPA, which only appeared in the late culture periods (FIG. 4, Keloid: upper panel).

Reverse fibrin overlay assay revealed that normal fibroblasts expressed PAI-1 in a variable activity level (FIG. 4, Normal: lower panel). In drastic contrast, keloid fibroblasts expressed a persistently high level of PAI-1 throughout the entire culture period (FIG. 4, Keloid: lower panel).

The PAI-1 activity was also measured using Chromogenic Substrate Assay (American Diagnostica). In the assay, keloid fibroblasts typically showed a 2- to 3-fold higher levels in PAI activity than normal fibroblasts (K:N, 45:10; 80:45; 40:16 IU/ml in 3 separate measurements). A small amount of uPA/PAI-1 complex was detected in cultures of both normal and keloid fibroblasts (FIG. 4, upper panels: uPA/PAI-1 complex). The complex was catalytically inactive in situ, and its fibrinolytic activity, which appeared in the fibrin overlay, was due to an artifact of SDS treatment during the SDS-PAGE procedure. Granelli-Piperno & Reich, A study of proteases and protease-inhibitor complexes in biological fluids, J. Exp. Med. 148: 223-234 (1978).

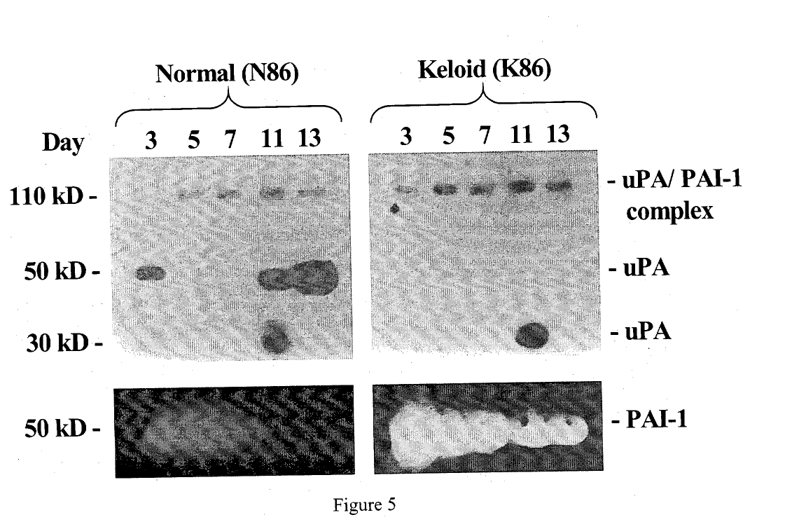

Activities of uPA and PA1 were also examined in a pair of donor- and anatomical site-matched samples, N86 and K86. The result is shown in FIG. 5. With a slight difference in the time and level of expression, N86 exhibited a very similar pattern of uPA expression (FIG. 5, N86: upper panel) when compared to other normal fibroblasts (FIG. 4, Normal: upper panel). N86 is different, however, in its PAI-1 expression, which appeared very high in the early half of the culture period and disappeared in the later half (FIG. 5, N86: lower panel). The expression patterns of uPA and PAI-1 by K86 were very similar to other keloid fibroblasts in which uPA of the 30 kD form appeared in a moderate amount at day 11, and PAI-1 was over-expressed throughout the entire culture period (FIG. 5, K86). The presence of the uPA/PAI-1 complex (˜110 kD) in keloid samples (FIGS. 4 & 5) indicated that the UPA secreted by keloid fibroblasts was largely bound by PAI-1. Therefore, in long term fibrin gel cultures, while normal fibroblasts exhibit regulated expressions of uPA and PAI-1, keloid fibroblasts exhibit a low level of uPA and a persistently high level of PAI-1. Furthermore, PAI-1 over-expression by keloid fibroblasts correlates with the elevated collagen accumulation.

The High PAI-1 Activity is Causal in Elevated Collagen Accumulation of Keloid Fibroblasts

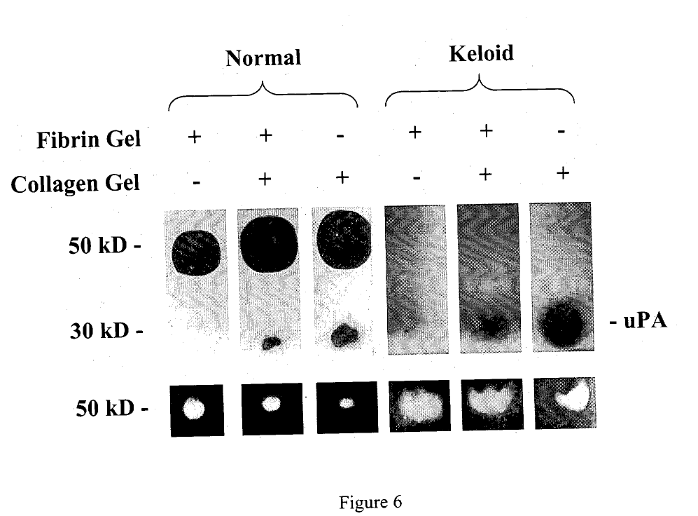

Fibroblasts in fibrin matrix actively reorganize the matrix and produce collagen to replace fibrin. Tuan et al., In vitro fibroplasia: matrix contraction, cell growth, and collagen production of fibroblasts cultured in fibrin gels, Exp. Cell Res. 223: 127-134 (1996). To determine if the expression pattern of uPA or PAI-1 by fibroblasts in fibrin gels was influenced by the changing extracellular matrix (ECM) environment (i.e., from fibrin to collagen), fibrin, fibrin-collagen, or collagen gels were used in cell cultures to mimic the matrix phenotype of early, mid, or late stage during in vitro fibroplasia. Results showed that in normal fibroblasts, uPA expression transitioned from the 50 kD two chain form to the 50 kD and 30 kD forms in the presence of collagen (fibrin-collagen and collagen gels) (FIG. 6, Normal, upper panels). Interestingly, the level of PAI-1 expression decreased as the concentration of collagen in the gel matrix increased from 50% to 100% (FIG. 6, Normal, lower panels). Keloid fibroblasts responded to the presence of collagen in the matrix by expressing 30 kD uPA; however, there was no significant change in their PAI-1 level (FIG. 6, Keloid). Therefore, while expressions of both uPA and PAI-1 of normal fibroblasts were modulated by ECM, only uPA expression of keloid fibroblasts was subjected to ECM modulation. There was no difference in cell growth among cultures of fibrin, collagen, or fibrin-collagen hybrid gels. Therefore, PAI-1 over-expression is an intrinsic characteristic of keloid fibroblasts, regardless of the level of collagen in ECM.

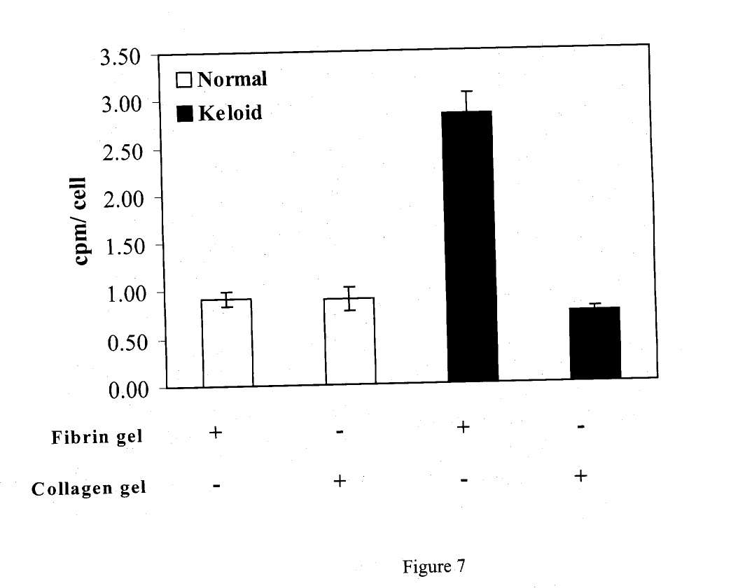

Collagen accumulation of normal or keloid fibroblasts was also studied and compared between cultures of fibrin and collagen gels. Results showed that the level of collagen accumulation by normal fibroblasts was similar between cultures of fibrin and collagen gels (FIG. 7, Normal). In contrast, when keloid fibroblasts were cultured in collagen gels, their usually high level of collagen accumulation observed in fibrin gels was reduced to a level comparable to normal fibroblasts (FIG. 7, Keloid). A similar reduction in collagen accumulation was found in keloid fibroblasts when they were cultured in fibrin-collagen gels. Similar data were obtained in two additional strains of keloid fibroblasts. These results indicate that the high PAI-1 activity is necessary in sustaining the elevated collagen accumulation by keloid fibroblasts, because an increase in uPA activity by culturing keloid cells in collagen or fibrin-collagen hybrid gels reduced collagen accumulation of keloid fibroblasts.

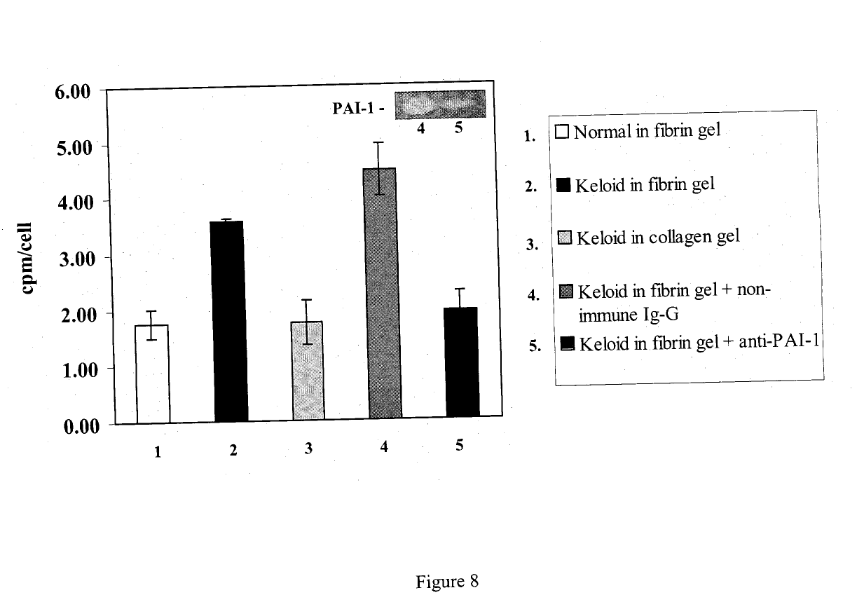

To further test if the high PAI-1 activity led to increased collagen accumulation, collagen accumulation of keloid fibroblasts in cultures of fibrin gels was studied in the presence of PAI-1 neutralizing antibodies. According to the manufacturer, the antibodies (rabbit anti-PAI-1 antibody; #395R, American Diagnostica) react with all forms of human PAI-1. At the 50% inhibition point, 1 mg of this antibody can inhibit ˜1000 IU of PAI-1. Results showed that anti-PAI-1antibodies, but not non-immune IgG, decreased PAI-1 activity (FIG. 8, insert) and reduced collagen accumulation of keloid fibroblasts (FIG. 8, “Keloid in fibrin gel+anti-PAI-1”). Two additional strains of keloid fibroblasts were also tested for the effect of anti-PAI-1 neutralizing antibodies on collagen accumulation. Studies of collagen accumulation in fibrin gel cultures of normal fibroblasts or collagen gel cultures of keloid fibroblasts were also conducted at the same time for comparison (FIG. 8, “Normal in fibrin gel” and “Keloid in collagen gel”).

Discussion

The examples in the present invention demonstrate that PAI-1 over-expression is a consistent feature of keloid fibroblasts both in vitro and in vivo. In long term fibrin gel cultures, while normal fibroblasts exhibit regulated levels of uPA and PAI-1 as well as collagen accumulation, keloid fibroblasts exhibit persistently high levels of PAI-1 and collagen accumulation. Conditions that would reduce PAI-1 activity abolish the elevated collagen accumulation of keloid fibroblasts. These conditions include increasing uPA by culturing fibroblasts in collagen or fibrin-collagen gels, or decreasing PAI-1 activity by adding PAI-1 neutralizing antibodies to fibroblasts in cultures of fibrin gels or other methods described herein. Therefore, the increased PAI-1 activity of keloid fibroblasts may account for their elevated collagen accumulation in fibrin gel cultures.

Fibroplasia is a dynamic process that incorporates constant interactions and feedbacks between participating cell, ECM, and soluble mediators. Clark, Wound Repair: Overview and General Considerations, The Molecular and Cellular Biology of Wound Repair, pp. 22-32 (Edited by Clark R A. New York, Plenum Press, 1996). It was previously shown that normal skin fibroblasts can actively reorganize the fibrin matrix and remodel it into a collagen-containing scar-like tissue. Tuan et al., In vitro fibroplasia: matrix contraction, cell growth, and collagen production of fibroblasts cultured in fibrin gels, Exp. Cell Res. 223: 127-134 (1996). From examples in the present invention, it is evident that as normal fibroblasts synthesize and deposite collagen into the fibrin matrix, the activity levels of uPA and PAI-1 are also regulated (FIGS. 4 and 5). This ECM-mediated change in uPA and PAI-1 expressions is proven in subsequent experiments using fibrin, fibrin-collagen mixture, or collagen gels (FIG. 6). Integrins are the likely candidates in mediating such dynamic reciprocity between fibroblasts and the ECM, because integrin engagement or disengagement from ECM may mediate an integrin species-specific change in the phenotype of cells. Xu & Clark, Extracellular matrix alters PDGF regulation of fibroblast integrins, J. Cell Biol. 132: 239-249 (1996). The evidence can be further drawn from studies of collagen gels. In collagen gels, the binding of α₂β₁ integrin to collagen increases cell survival and ECM production; in contrast, the disruption of α₂β₁ binding to collagen induces MMP2 production/activation, therefore,—matrix degradation. Ellerbroek et al., Functional interplay between type I collagen and cell surface matrix metalloproteinase activity, J. Biol. Chem. 276: 24833-24842 (2001). It has been shown that fibroblasts are able to bind to fibrin using integrins containing the αᵥ subunit. Gailit et al., Human fibroblasts bind directly to fibrinogen at RGD sites through integrin alpha(v)beta3, Exp. Cell Res. 232: 118-126 (1997). It is, however, not excluded in the current study that fibronectin may be involved in the binding of fibroblasts to the fibrin gel matrix through α₅β₁ integrin. Clark, Wound Repair: Overview and General Considerations, The Molecular and Cellular Biology of Wound Repair, pp. 22-32 (Edited by Clark R A. New York, Plenum Press, 1996), because fibrinogen used in the study contains a trace amount of fibronectin (<0.1 μg/mg of fibrinogen), and 10% FCS (which contains fibronectin) was used in the collagen synthesis assay. Therefore, the difference in uPA and PAI-1 expression between fibrin and collagen gels may be mediated by a difference in αᵥ-containing integrin or α₅β₁ binding to fibrin/fibronectin and/or α2β₁ binding to collagen.

Increased PAI-1 activity has been a hallmark of tissue and organ fibrosis. There is evidence that a direct correlation exists between the genetically determined level of PAI-1 expression and the extent of collagen accumulation that follows inflammatory lung injury. The support was drawn from studies of bleomycin-induced pulmonary fibrosis in transgenic mice. Eitzman et al., Bleomycin-induced pulmonary fibrosis in transgenic mice that either lack or overexpress the murine plasminogen activator inhibitor-1 gene, J. Clin. Invest. 97: 232-237 (1996). These studies were based on the rationale that excessive PAI-1 activity leads to fibrin accumulation, which in turn elicits a fibrogenic effect on lung repair. Fibrin is the best-known substrate of plasmin and its breakdown products are chemotactic to inflammatory cells. Clark, Wound Repair: Overview and General Considerations, supra. Therefore, accumulation of fibrin at the site of tissue injury is causal for tissue fibrosis. In addition, the difference in the uPA:PAI-1 ratio between normal and keloid fibroblasts was reflected in the degree of fibrin matrix degradation, whereas, in a short term assay, normal fibroblasts caused fibrin matrix degradation but keloid fibroblasts did not. Tuan et al., Elevated levels of plasminogen activator inhibitor-1 may account for the altered fibrinolysis by keloid fibroblasts, J. Invest. Dermatol. 106: 1007-1011 (1996). Furthermore, the treatment of normal fibroblasts with TGF-β, a potent inducer of PAI-1(Keski-Oja et al., Regulation of mRNAs for type-1 plasminogen activator inhibitor, fibronectin and type I procollagen by transforming growth factor-beta. Divergent responses in lung fibroblasts and carcinoma cells, J. Biol. Chem. 263: 3111-3115 (1988)), prevented fibrin degradation. Clinical observations have revealed that before keloids form the affected area is preceded by a prolonged inflammatory reaction. In addition, most keloids have three distinctive areas: an erythematous outer border (area of expansion/growth), an inner non-erythematous raised border (classical keloid), and a central regressing area. As fibrin is involved in inflammation, it is believed in the art that keloid lesions, especially the outer border, may contain greater accumulations of fibrin.

Nevertheless, the notion of fibrin as a cause for fibrosis has been challenged recently in lung injury-repair studies. In mutant mice lacking a α or γ chain of fibrinogen and with no intact fibrinogen in the circulation, the degree of lung fibrosis after bleomycin treatment was comparable to the wild type mice. Wilberding et al., Development of pulmonary fibrosis in fibrinogen-deficient mice, Ann. N. Y Acad. Sci. 936: 542-548 (2001). These studies indicate that while fibrin may promote fibrosis, it does not appear to be a pre-requisite of fibrosis. From the examples of the present invention, the reduction of collagen accumulation of keloid fibroblasts by adding PAI-1 neutralizing antibody into fibrin cultures or by culturing cells in fibrin-collagen or collagen gels (which induces uPA expression), strongly suggests that the over-expression of PAI-1, instead of fibrin, may be the key to excessive collagen accumulation in keloid fibrosis. The fact that both PAI-1 over-expression (FIG. 2) (see also Tuan et al., Elevated levels of plasminogen activator inhibitor-1 may account for the altered fibrinolysis by keloid fibroblasts, J. Invest. Dermatol. 106: 1007-1011 (1996); Higgins et al., Differential regulation of PAI-1 gene expression in human fibroblasts predisposed to a fibrotic phenotype, Exp. Cell Res. 248: 634-642 (1999) and collagen overproduction; Uitto et al., Altered steady-state ratio of type VIII procollagen mRNAs correlates with selectively increased type I procollagen biosynthesis in cultured keloid fibroblasts, Proc. Natl. Acad. Sci. U.S.A. 82: 5935-5939 (1985)) have been found in cultures of keloid fibroblasts on plain cell culture surfaces in the absence of fibrin, gives further support of the involvement of PAI-1 in keloid fibrosis.

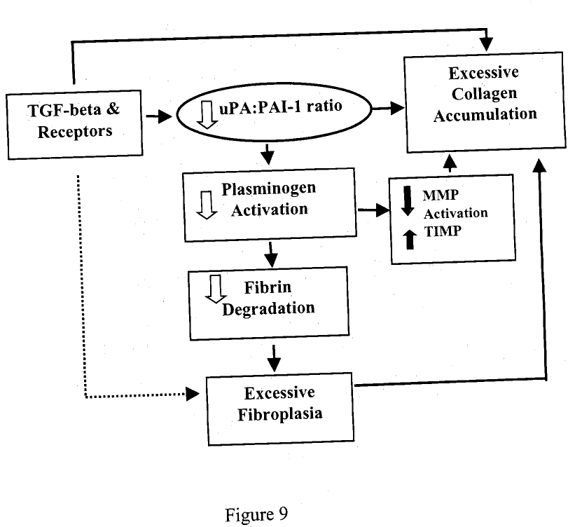

It is noteworthy that the collagen purification protocol employed in the examples of this invention by pepsin treatment recovers only intact collagen and reflects collagen accumulation. Epstein, Alpha1-3 human skin collagen. Release by pepsin digestion and preponderance in fetal life, J. Biol. Chem. 249: 3225-3231 (1974). Since collagen production may be modulated post-translationally by proteases of the matrix metalloproteinase (MMP) family (Rossert & Crombrugghe, Structure, Synthesis, and Regulation of Type I Collagen. Principles of Bone Biology, San Diego Academic Press, pp. 127-142 (1996)), it is possible that the reduction of collagen accumulation by keloid fibroblasts cultured in collagen or fibrin-collagen gels is due to collagen degradation caused by plasmin-mediated MMP activation (pathways summarized in FIG. 9). Alternatively, an integrin-mediated mechanism maybe involved, since PAI-1, aside from its effect on cell growth and apoptosis, is able to modulate integrin-mediated cell adhesion and migration through its binding to uPA and to vitronectin. Stefansson & Lawrence, The serpin PAI-1 inhibits cell migration by blocking integrin alpha V beta 3 binding to vitronectin, Nature 383: 441-443 (1996). Therefore, the change in uPA: PAI-1 ratio of keloid fibroblasts under conditions mentioned above might affect the binding of keloid fibroblasts to the gel-matrix and, subsequently, alter the state of fibroblast differentiation and collagen synthesis. Ellerbroek et al., Functional interplay between type I collagen and cell surface matrix metalloproteinase activity, J. Biol. Chem. 276: 24833-24842 (2001); Streuli, Extracellular matrix remodelling and cellular differentiation, Curr. Opin. Cell Biol. 11: 634-640 (1999). These motions can be further tested by employing the in vitro fibroplasia model.

When cultured in collagen gels, it was interesting to note that while normal fibroblasts exhibited an increase in uPA and a decrease in PAI-1 levels, there was no change in the levels of collagen accumulation (FIG. 6 and 7). This might be due to the isometric tension developed in the matrix during fibroblast contraction of gels. It has been previously shown that the isometric tension developed in the ECM matrix as a result of cell-matrix interaction may dictate the metabolic state of the cell. Nakagawa et al., Extracellular matrix organization modulates fibroblast growth and growth factor responsiveness, Exp. Cell Res. 182: 572-582 (1989). Accordingly, fibroblasts in collagen gels detached from the tissue culture dish, allowing the fibroblasts to contract the collagen matrix under relatively little tension, produce very little collagen. In contrast, as fibroblasts in attached collagen gels contract the matrix and generate increasing tension, the basal collagen synthesis is maintained. Nakagawa et al., supra. In examples of the present invention, both fibrin and collagen gels were attached to culture dishes; therefore, no difference in collagen accumulation was observed.

The epidermis of keloids also showed a stronger PAI-1 expression than that of normal skin and normal scar (FIG. 1). This may also have some clinical implications since human adult keratinocytes do not normally express PAI-1. Its expression accompanies epidermal migration and only occurs during wound repair. Li et al., Targeted inhibition of wound-induced PAI-1 expression alters migration and differentiation in human epidermal keratinocytes, Exp. Cell. Res. 258: 245-253 (2000). Other serine or MMP protease inhibitors such as alpha-1 antitrypsin, alpha-2 macroglobulin, and tissue alpha-globulins were also detected in keloid lesions. Diegelmann et al., Tissue alpha-globulins in keloid formation, Plast. Reconstr. Surg. 59: 418-423 (1977). The effect of these proteins on keloid fibroplasias can also be tested in the future employing the in vitro model system. In conclusion, using the three dimensional matrix gel systems, the examples of the present invention demonstrated that PAI-1 over-expression correlates with elevated collagen accumulation by keloid fibroblasts. When PAI-1 activity is inhibited or reduced, the abnormal collagen accumulation is abolished, thus proving a causal relationship between the two. A schematic diagram depicting the major findings in keloid fibrosis and connecting them to key events/components of tissue injury repair is presented in FIG. 9.

Papers and patents cited in the disclosure are expressly incorporated by reference in their entireties. It is to be understood that the description, specific examples, and figures, while indicating preferred embodiments, are given by way of illustration and exemplification an are not intended to limit the present invention. Various changes and modifications within the present invention will become apparent to the skilled artisan from the disclosure contained herein. Therefore, the spirit and scope of the appended claims should not be limited to the description of the preferred versions contained herein.

TABLE 1
Normal skin, scar, and keloid fibroblasts used in the study

| | | Ethnic | | | Anatomical |
|---|---|---|---|---|---|
| Type | Cell Strain | Background | Gender | Age | Site |
| Normal | N65 | Hispanic | M | 8 | Ear |
| Skin | | | | | |
| Normal | N77 | African- | M | 4 | Ear |
| Skin | | American | | | |
| Normal | N86 | African- | M | 28 | Ear |
| Skin | | American | | | |
| Normal | N123 | Caucasian | F | 23 | NK* |
| Skin | | | | | |
| Normal | N127 | Iranian | M | 33 | Post ear |
| Skin | | | | | |
| Normal | N141 | Caucasian | M | Newborn | Foreskin |
| Skin | | | | | |
| Normal | N143 | Caucasian | M | Newborn | Foreskin |
| Skin | | | | | |
| Normal | N144 | Caucasian | M | Newborn | Foreskin |
| Skin | | | | | |
| Normal | K7N | African- | NK* | NK* | NK* |
| Skin | | American | | | |
| Normal | NSC14 | Hispanic | F | 25 | Elbow |
| Scar | | | | | |
| Normal | NS70 | Caucasian | M | 8 | Foot |
| Scar | | | | | |
| Normal | NS75 | African- | M | 4 | Cheek |
| Scar | | American | | | |
| Keloid | K9 | African- | F | NK* | Ear lobe |
| | | American | | | |
| Keloid | K10 | African- | F | NK* | Flank |
| | | American | | | |
| Keloid | K74 | African- | M | 28 | Ear lobe |
| | | American | | | |
| Keloid | K76 | African- | M | 4 | Ear |
| | | American | | | |
| Keloid | K80 | Hispanic | M | 5 | Supra-pubic |
| Keloid | K86 | African- | M | 28 | Ear |
| | | American | | | |
| Keloid | K109 | African- | M | 14 | Ear |
| | | American | | | |
| Keloid | K134 | Asian | F | 6 | Chest |
| Keloid | K135 | Hispanic | F | 6 | Finger |
| Keloid | K139 | African- | F | 47 | Ear |
| | | American | | | |
| Keloid | K142 | African- | F | 7 | Sternal area |
| | | American | | | |
| Keloid | K147 | African- | M | 18 | Ear |
| | | American | | | |
| Keloid | K148 | African- | M | 14 | Ear |
| | | American | | | |
| Keloid | K150c | African- | F | 23 | Chest |
| | | American | | | |
| Keloid | K165 | Hispanic | M | 5 | Ear |

*NK = not known

What is claimed is:

1. A method of reducing an excessive accumulation of collagen in a wound healing process comprising the step of reducing a PAI-1 activity.

2. The method of claim 1 wherein the PAI-1 activity is reduced by a PAI-inhibitor.

3. The method of claim 2 wherein the PAI-1 inhibitor is an indirect PAI-1 inhibitor or a direct PAI-1 inhibitor.

4. The method of claim 3 wherein the indirect PAI-1 inhibitor is selected from the group consisting of Fosinopril; Imidapril; Captopril; Enalapril; L158,809; Eprosartan; Troglitazone; Vitamin C; Vitamin E; Perindorpril; Mifepristone (RU486); Spironolactone; and a RCL peptide.

5. The method of claim 3 wherein the direct PAI-1 inhibitor is selected from the group consisting of PAI-1 neutralizing antibodies, diketopiperazine based compounds, tetramic acid based compounds, hydroxyquinolinone based compounds, and 11-keto-9(E), 12(E)-octadecadienoic acid.

6. The method of claim 2 wherein the PAI-1 inhibitor is administered to a subject undergoing the wound healing process.

7. The method of claim 6 wherein the PAI-1 inhibitor is administered by a route selected from the group consisting of an epidermal administration, a transdermal administration, a pulmonary administration, a nasal administration, an ophthalmic administration, a buccal administration, an oral administration, a rectal administration, a vaginal administration, and a parenteral administration.

8. The method of claim 1 wherein the excessive accumulation of collagen leads to an abnormal scar selected from the group consisting of a keloid, an adhesion, a hypertrophic scar, a skin disfiguring condition, a fibrosis, a fibrocystic condition, a contracture, a scleroderma, a Duypuytren's disease, a Peyronie's disease, and a joint stiffness.

9. The method of claim 8 wherein the fibrosis is selected from the group consisting of intersticial fibrosis, kidney fibrosis, liver fibrosis, pulmonary fibrosis, cardiac fibrosis, and retinal and vitreal retinopathy.

10. A method of preventing the formation of an abnormal scar that results from an excessive accumulation of collagen comprising the step of reducing a PAI-1 activity.

11. The method of claim 10 wherein the PAI-1 activity is reduced by a PAI-inhibitor.

12. The method of claim 11 wherein the PAI-1 inhibitor is an indirect PAI-1 inhibitor or a direct PAI-1 inhibitor.

13. The method of claim 12 wherein the indirect PAI-1 inhibitor is selected from the group consisting of Fosinopril; Imidapril; Captopril; Enalapril; L158,809; Eprosartan; Troglitazone; Vitamin C; Vitamin E; Perindorpril; Mifepristone (RU486); Spironolactone; and a RCL peptide.

14. The method of claim 12 wherein the direct PAI-1 inhibitor is selected from the group consisting of PAI-1 neutralizing antibodies, diketopiperazine based compounds, tetramic acid based compounds, hydroxyquinolinone based compounds, and 11-keto-9(E), 12(E)-octadecadienoic acid.

15. The method of claim 11 wherein the PAI-1 inhibitor is administered to a subject wherein the excessive accumulation of collagen is observed in the subject.

16. The method of claim 15 wherein the PAI-1 inhibitor is administered by a route selected from the group consisting of an epidermal administration, a transdermal administration, a pulmonary administration, a nasal administration, an ophthalmic administration, a buccal administration, an oral administration, a rectal administration, a vaginal administration, and a parenteral administration.

17. The method of claim 10 wherein the abnormal scar is selected from the group consisting of a keloid, an adhesion, a hypertrophic scar, a skin disfiguring condition, a fibrosis, a fibrocystic condition, a contracture, a scleroderma, a Duypuytren's disease, a Peyronie's disease, and a joint stiffness.

18. The method of claim 17 wherein the fibrosis is selected from the group consisting of intersticial fibrosis, kidney fibrosis, liver fibrosis, pulmonary fibrosis, cardiac fibrosis, and retinal and vitreal retinopathy.

19. A method of treating an abnormal scar that results from an excessive accumulation of collagen comprising the step of reducing a PAI-1 activity.

20. The method of claim 19 wherein the PAI-1 activity is reduced by a PAI-inhibitor.

21. The method of claim 20 wherein the PAI-1 inhibitor is an indirect PAI-1 inhibitor or a direct PAI-1 inhibitor.

22. The method of claim 21 wherein the indirect PAI-1 inhibitor is selected from the group consisting of Fosinopril; Imidapril; Captopril; Enalapril; L158,809; Eprosartan; Troglitazone; Vitamin C; Vitamin E; Perindorpril; Mifepristone (RU486); Spironolactone; and a RCL peptide.

23. The method of claim 21 wherein the direct PAI-1 inhibitor is selected from the group consisting of PAI-1 neutralizing antibodies, diketopiperazine based compounds, tetramic acid based compounds, hydroxyquinolinone based compounds, and 11-keto-9(E), 12(E)-octadecadienoic acid.

24. The method of claim 20 wherein the PAI-1 inhibitor is administered to a subject having the abnormal scar.

25. The method of claim 24 wherein the PAI-1 inhibitor is administered by a route selected from the group consisting of an epidermal administration, a transdermal administration, a pulmonary administration, a nasal administration, an ophthalmic administration, a buccal administration, an oral administration, a rectal administration, a vaginal administration, and a parenteral administration.

26. The method of claim 19 wherein the abnormal scar is selected from the group consisting of a keloid, an adhesion, a hypertrophic scar, a skin disfiguring condition, a fibrosis, a fibrocystic condition, a contracture, a scleroderma, a Duypuytren's disease, a Peyronie's disease, and a joint stiffness.

27. The method of claim 26 wherein the fibrosis is selected from the group consisting of intersticial fibrosis, kidney fibrosis, liver fibrosis, pulmonary fibrosis, cardiac fibrosis, and retinal and vitreal retinopathy.

28. A method of determining the propensity of the formation of an abnormal scar comprising the steps of
a) locating a wound site; and
b) measuring the level of a PAI-1 activity.

29. The method of claim 28 further comprising steps of comparing the PAI-1 activity with a standard PAI-1 activity and determining the likelihood of forming the abnormal scar.

30. The method of claim 28 wherein the level of the PAI-1 activity is measured by an ELISA, a chromogenic assay, a fibrin overlay assay, or a reverse fibrin overlay assay.

31. The method of claim 28 wherein the abnormal scar is a keloid, an adhesion, a hypertrophic scar, a skin disfiguring condition, a fibrosis, a fibrocystic condition, a contracture, a scleroderma, a Duypuytren's disease, a Peyronie's disease, and a joint stiffness.

32. The method of claim 31 wherein the fibrosis is selected from the group consisting of intersticial fibrosis, kidney fibrosis, liver fibrosis, pulmonary fibrosis, cardiac fibrosis, and retinal and vitreal retinopathy.
